(19) United States
(12) Patent Application Publication
(10) Pub. No.: US 2013/0218042 A1
(43) Pub. Date: Aug. 22, 2013

(54) SYSTEMS AND/OR METHODS FOR STIMULATING THE BRAIN TO PROMOTE LEARNING AND/OR TO PROVIDE THERAPEUTIC TREATMENTS THERETO

(71) Applicant: Aris Mardirossian, Potomac, MD (US)

(72) Inventor: Aris Mardirossian, Potomac, MD (US)

(73) Assignee: Patents Innovations, LLC, Venice, FL (US)

(21) Appl. No.: 13/402,042

(22) Filed: Feb. 22, 2012

(51) Int. Cl. A61N 1/36; A61B 5/0476

(52) U.S. Cl. 600/544; 607/45

(57) ABSTRACT

Certain example embodiments of this invention relate to systems and/or methods for stimulating the brain, e.g., to promote learning and/or memory, to provide therapeutic treatments thereto, etc. In certain example embodiments, neurons are monitored and their activities are analyzed to determine the electrical and electrochemical pathways that are activated during specific learning functions, events, etc. Given an established mapping, certain example embodiments deliver electrical and electrochemical signals to specifically targeted brain locations in order to facilitate learning and/or memory, to provide therapeutic treatments thereto, etc. For example, neurons or gateways thereto may be stimulated to facilitate learning, memory and/or recall, to counteract or mitigate the onset or arrival of mental or neurological diseases and disorders, etc.

### FIELD OF THE INVENTION

Certain example embodiments of this invention relate to techniques for assisting the ways in which subjects receive and process information. More particularly, certain example embodiments of this invention relate to systems and/or methods for stimulating the brain, e.g., to promote learning and/or memory, to provide therapeutic treatments thereto, etc. In certain example embodiments, neurons are monitored and their activities are analyzed to determine the electrical and electrochemical pathways that are activated during specific learning functions. Given an established mapping, certain example embodiments deliver electrical and electrochemical signals to specifically targeted brain locations in order to facilitate learning and/or memory.

### BACKGROUND AND SUMMARY OF EXAMPLE EMBODIMENTS OF THE INVENTION

Humans have been learning in the same basic ways for thousands of years. For instance, humans receive a variety of visual, aural, tactile, olfactory, and even taste-related stimuli. These stimuli are internalized and, based on such experiences, humans gradually learn and develop reasoning skills.

Various techniques have developed, e.g., to help humans master traditional “reading, writing, and arithmetic” skills, and different approaches have developed over time to hone these basic skills. For instance, to promote better reading skills, approaches like phonics, the Stevenson method, etc., have been developed. Similarly, various learning “devices” (including mnemonic devices) have been developed to help promote short- and long-term memory. Fundamentally, however, information and experience must be made available before it can be consumed, either willingly or unintentionally, by a person.

The development of the printing press resulted in an explosion of information that changed the course of history. More recently, the advent of the information age, including the ubiquitous nature of computer networks like the Internet, has made even more information available even faster to a potentially broader cross-section of information consumers. These and other intervening vehicles have transformed the way that information and experience is delivered and consumed.

Yet with all of these advances in the way that information and experience can be delivered, the same basic senses (sight, sound, touch, smell, and/or taste) are still the primary ways in which humans actually ingest the information and experiences. That information and those experiences still must be process once ingested. The transformations in the way that information is proliferated, coupled with the lack of transformations in the ways that information is absorbed, have in some cases promoted “information overload.” Indeed, people generally cannot ingest and process the huge amount of information that is readily available to them, e.g., with only the click of a button or the poking of a computer key.

Thus, it will be appreciated that there is a need for techniques that transform the way that people take in and/or process information. It also will be appreciated that there is a need for techniques that reveal on a fundamental level how information is taken in and/or processed.

In certain example embodiments, a brain activity monitoring method is provided. A group of test subjects is selected such that the test subjects have at least some personal attributes in common. The group of test subjects is asked or allowed to perform a common event over a predetermined time period. Electrical signals generated by individual test subjects are measured as each performs the common event. The measured electrical signals are attributed to neurons and/or neural pathways of the test subjects' brains to develop a time-indexed map of neural activity for the common event for each said test subject, with the attributing including assigning location and intensity values to each measured electrical signal at regular intervals over the predetermined time period during which the common event was performed. The maps are compared to one another and outlier maps, locations, and intensity values, are discarded. One or more maps are stored to a database of a computer system as a template time-indexed map of neural activity for the common event.

In certain example embodiments, a brain activity monitoring system is provided. Measuring means measure electrical signals generated by individual test subjects in a group of test subjects as each performs a common event over a predetermined time period, with the test subjects being selected so that they have at least some personal attributes in common. A non-transitory storage location stores data generated by the measuring means. A computer system includes at least one processor and a memory and is configured to: (a) read the data generated by the measuring means stored in the storage location, (b) attribute the measured electrical signals to neurons and/or neural pathways of the test subjects' brains to develop a time-indexed map of neural activity for the common event for each said test subject, the attributing including assigning location and intensity values to each measured electrical signal at regular intervals over the predetermined time period during which the common event was performed, and (c) compare the maps to one another and discard outlier maps, locations, and intensity values. A database is in communication with the computer system and is configured to store one or more maps as a template time-indexed map of neural activity for the common event.

According to certain example embodiments, key neurons or clusters of neurons associated with the common event may be identified, and/or one or more gateways in the brain to the identified key neurons or clusters of neurons may be identified.

According to certain example embodiments, brain activity may be monitored using fMRI and/or EEG hardware for a first subset of test subjects as the first sub-set of test subjects performs the common event, and one or more portions of the brain that appear(s) to be associated with the common event based on output from the fMRI and/or EEG hardware may be identified. In some cases, the measuring of the electrical signals may be focused on the one or more identified portions of the brain, and/or the measuring may be performed on a second subset of test subjects following said monitoring.

According to certain example embodiments, the personal attributes may include one or more of: age, education level, ethnicity, gender, mental disease and/or disorder, nervous system malady, and race.

According to certain example embodiments, the common event may be selected from the group consisting of: reading, writing, performing mathematical operations, memorizing, suffering a seizure, and engaging a muscle or group of muscles.

In certain example embodiments, a method of stimulating a subject's brain is provided. A template map of neural activity is selected from a database comprising a plurality of template maps. The template map is selected based at least in part on a desired treatment objective and subject-specific characteristics of the subject. The template map indicates neurons and/or neural pathways to be electrically excited, and an intensity of excitation for each, at specific times over a predefined treatment time period. The subject's brain is electrically stimulated in accordance with the selected template map.

In certain example embodiments, a treatment device is provided. A processor is configured to read a template map indicating neurons and/or neural pathways to be electrically excited, and an intensity of excitation for each, at specific times over a predefined treatment time period. The template map represents a desired treatment objective for a subject and the treatment map being customized for associated subject-specific characteristics. An electrode array includes one or more electrode and is configured to electrically stimulating the subject's brain in accordance with the selected template map, based on instructions from the processor.

According to certain example embodiments, the desired treatment objective may include one or more of: an educational goal, memory/recall improvement, nerve reconditioning, and mitigation and/or correction of neural degradation.

According to certain example embodiments, during the electrical stimulating, an electromagnetic field is set up in/on the subject's brain in accordance with the template map via electrodes external to the subject's body. In some cases, the intensities in the template map may be reduced from measured values by a predetermined percentage. For instance, in some cases, the intensities in the template map may be reduced to levels below threshold levels for directly initiating synaptic responses.

The features, aspects, advantages, and example embodiments described herein may be combined to realize yet further embodiments.

### BRIEF DESCRIPTION OF THE DRAWINGS

These and other features and advantages may be better and more completely understood by reference to the following detailed description of exemplary illustrative embodiments in conjunction with the drawings, of which:

FIG. 1 is a schematic view of a neuron;

FIG. 2 is a flowchart showing a process for gathering information concerning the parts of a brain that are activated during specific events of interest, in accordance with certain example embodiments;

FIG. 3a is an example Brainbow-type image that may be used in connection with certain example embodiments;

FIG. 3b is an example diffusion spectrum imaging (DSI) type image that may be used in connection with certain example embodiments;

FIG. 4 is a table showing intensity measurements of various nodes for five test subjects at a given point in time, showing one type of data and how it can be recorded in connection with certain example embodiments;

FIG. 5 is a graph plotting the FIG. 4 data;

FIG. 6 is an example data structure showing connections between nodes at a given time and for a given test subject, thereby illustrating another type of data that may be gathered and analyzed in connection with certain example embodiments;

FIG. 7 is a flowchart showing a treatment process in accordance with certain example embodiments; and

FIG. 8 is a schematic view of a treatment device in accordance with certain example embodiments.

### DETAILED DESCRIPTION OF EXAMPLE EMBODIMENTS OF THE INVENTION

Certain example embodiments of this invention relate to systems and/or methods for stimulating the brain, e.g., to promote learning and/or memory, to provide therapeutic treatments thereto, etc. In certain example embodiments, neurons are monitored and their activities are analyzed to determine the electrical and electrochemical pathways that are activated during specific learning functions. Given an established mapping, certain example embodiments deliver electrical and electrochemical signals to specifically targeted brain locations in order to facilitate learning and/or memory, to provide therapeutic treatments thereto, etc.

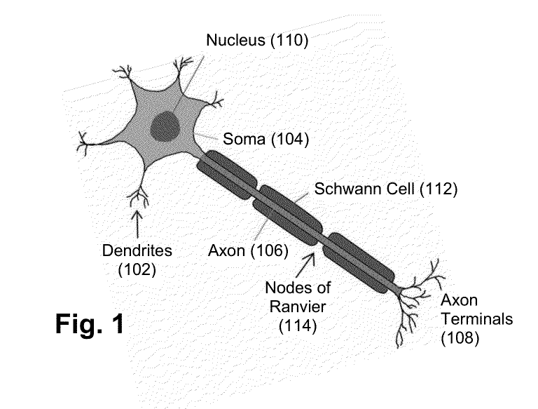

Referring now more particularly to the drawings in which like reference numerals indicate like parts throughout the several views, FIG. 1 is a schematic view of a neuron. A neuron processes and transmits information by electrical and chemical signaling. A neuron thus is an electrically excitable cell that maintains a voltage gradient across its membrane via metabolically driven ion pumps and typically includes one or more dendrites 102, a single soma 104, a single axon 106, and one or more axon terminals 108. It is noted that not all neurons include axons. Dendrites 102 are synapses, and the protrusions thereof capture neurotransmitters released by the pre-synaptic neuron. Thus, it will be appreciated that it is generally here where the synapses from two (or more) neurons communicate with one another. The soma 104 (or cell body) houses the nucleus 110 of the neuron, and the surface of the soma 104 includes voltage-activated ion channels that help transmit signals generated by the dendrites 102. The area emerging from the soma 104 is known as the axon hillock, which includes a high concentration of voltage-activated sodium channels, where generated signals tend to converge. The axon hillock gives way to the axon 106, which is a thin and generally tubular protrusion that extends away from the soma 104. The axon 106 is insulated by a myelin sheath comprising either Schwann cells 112 or oligodendrocytes. The myelin sheath wraps around axon segments, forming a fatty layer that helps reduce the likelihood of ions entering or escaping from the axon 106. Nodes of Ranvier 114 can be found at gaps between adjacent myelin sheaths and help boost signals from the dendrites 102 towards the axon terminals 108. The axon terminals form a second class of synapses that have voltage-activated calcium channels that are implicated when signaling other neurons.

The ion pumps in a neuron combine with ion channels in the membrane to generate intracellular versus extracellular concentration differences of ions (including, for example, sodium, potassium, chloride, and calcium). Changes in the cross-membrane voltage can alter the function of voltage-dependent ion channels and, if the voltage changes by a sufficiently large amount, an action potential is generated. The action potential is an electrochemical pulse that travels along the axon and activates synaptic connections with other cells upon arrival. Neurons connect to form neural networks.

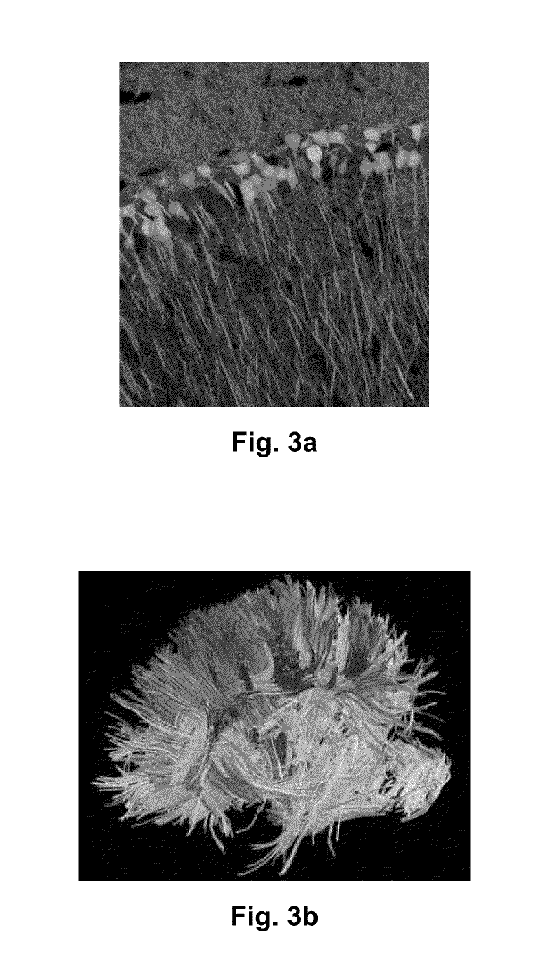

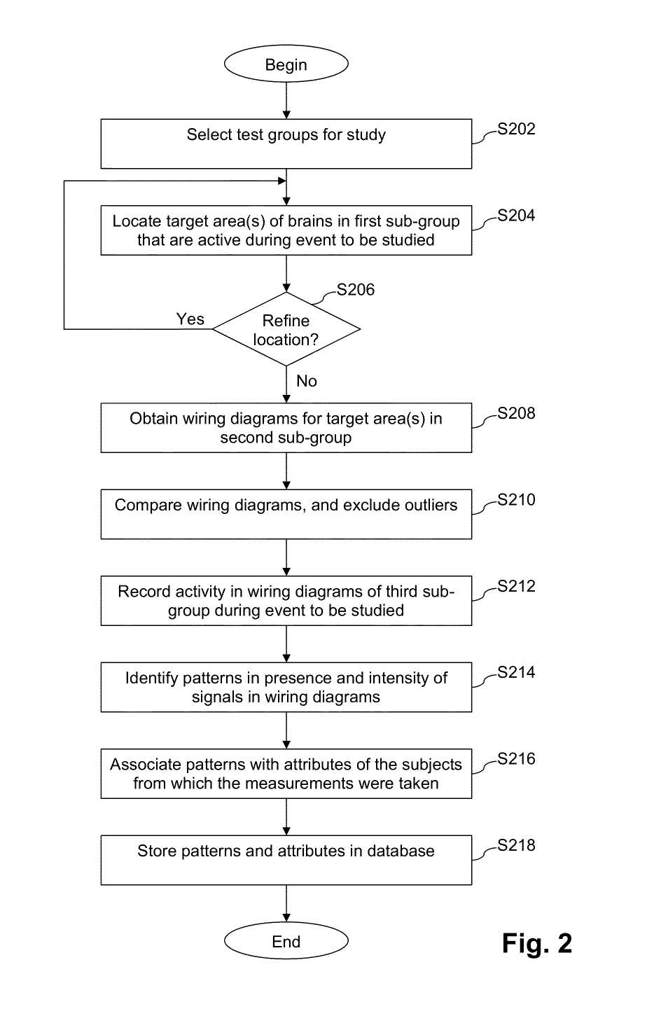

FIG. 2 is a flowchart showing a process for gathering information concerning the parts of a brain that are activated during specific events of interest, in accordance with certain example embodiments. In step S202, a test group is selected for study and the test group is broken down into plural sub-groups. The test group may include subjects having like characteristics. For instance, the test group may be of the same or similar age, gender, race, family background, educational level, and general worldly experience. Doing so may help reduce the number of variables associated with the study. If twins, triplets, or other multiples are involved, it may be desirable to ensure that they are in different sub-groups, e.g., to create paired samples during different phases of the study.

Depending on the event to be studied, physical characteristics of the brain may be considered. For instance, if the way in which an autistic child learns to speak is to be studied, then the test group might be certain to include a mix of known autistic and known non-autistic children. In certain example embodiments, it may be desirable to screen the test group to make sure that certain structural abnormalities with a brain are or are not present. This broad-based analysis may be accomplished using computer axial tomography (CAT) scans or structural magnetic resonance imaging (MRI) techniques, for example.

It may be necessary or desirable to locate more specific areas of the brain that are involved with the event to be studied. That is, it may be necessary or desirable to identify which specific portions of the brain are involved in the event to be studied, whether that event is learning to read, type, or how to tie one's shoes; practicing “mental math”; memorizing a poem; recalling what one ate for breakfast or did on a particular significant day or date; drawing or painting with a particular medium; taking good photographs; suffering from a stroke, seizure, bout of multiple sclerosis, Parkinson's, etc.; or undertaking any other particular event.

Some functions of the brain are known or believed to be known. For instance, it is believed that the parietal lobe is largely responsible for visuo-spatial skills, mathematics, and logic, whereas the temporal lobe is believed to be largely responsible for learning and memory, the visual cortex is believed to be largely responsible for vision, and the frontal lobe is believed to be largely responsible for planning, inhibition, and self-control. The corpus callosum is responsible for information transfer between the left and right sides of the brain. However, it may be necessary or desirable to further narrow-down the list of likely spatial candidates concerning where neurons are activated during the event to be studied.

In this regard, in step S204, the first sub-group is used to help locate target area(s) of the brain involved in the event to be studied. Relatively coarse locations of potential activity can be detected using electroencephalography (EEG), which helps indicate electrically active locations in the brain using detectors implanted in the brain or worn on a cap. In addition, or in the alternative, functional MRI (fMRI) can be used to derive images of brain activity. That is, such data may be gathered as members of the first sub-group complete the task to be studied. The areas that “light up” or otherwise seem active may be considered the target areas of interest and thus deserving of further study, e.g., of the type indicated below.

Inasmuch as the brain is the center of the nervous system in all vertebrate and most invertebrate animals and in a typical human includes 10s of billions of neurons, each connected by synapses typically to several thousand other neurons, it may be necessary to refine and further refine the locations of interest. Doing so may make the mapping operations discussed below more tractable. Thus, in step S206, a decision is made as to whether the location(s) of interest are suitably located. If not, then the location is to be refined, and the process may be returned to step S204 for further, more detailed tests to pinpoint one or more target areas to be studied, using the above-described and/or other techniques.

Once the target area(s) has/have been located or otherwise has been determined, “wiring diagrams” of a second sub-group are generated in step S208. Wiring diagrams, or connectomes, may reflect the connections between the neurons in an intact brain. A second sub-group, different from the first sub-group, may be used for this step in certain example scenarios. This is because the act of identifying the target area(s) of interest as discussed above may itself change the wiring diagram and thus provide false or misleading information as to which neurons are implicated in the event to be studied. That is, during step S204, the first sub-group may be engaged in the act of learning something for a first time. If the same first sub-group is used for subsequent steps, then the event to be monitored may be more of a recollection-type event rather than an initial learning type event. However, if twins, triplets, or other multiples are used with one (or more) being in the first sub-group and one (or more) being in the second sub-group, then better correlations potentially may be derivable, as multiples typically will have similar life experiences and common genetics. Indeed, some brain scan studies suggest that the majority of the quality of the fatty tissue that insulates axons is largely genetically determined. These connectomes may be considered pre-treatment connectomes.

There are various techniques that may be used, in vivo, for developing connectomes. There is, for example, the so-called Brainbow technique, which labels every neuron in a live brain a different color. By generating images of the brain, it can be seen where and how neurons connect to each other. FIG. 3a is an example Brainbow-type image that may be used in connection with certain example embodiments. Multiple images may be taken and subsequently refined using computer graphics image processing techniques to improve the quality and/or make “educated guesses” about neurons and connections, given that the initial quality may be lower given the fact that in vivo measurements are desirable. It is noted that Brainbow techniques generate random or pseudo-random coloration patterns. Thus, computer comparison techniques may be employed to detect color patterns and derive a consistent coloration or mapping across several connectomes, here and/or elsewhere in the example techniques described herein.

Diffusion spectrum imaging (DSI), which uses magnetic resonance signals to track the movement of water molecules in the brain, may be used as an alternative or additional technique. Because water diffuses along the length of axons, diffusion measurements can be used to map the “wires,” essentially creating a detailed blueprint of the brain's connectivity. FIG. 3b is an example diffusion spectrum imaging type image that may be used in connection with certain example embodiments. As above, computer graphics image processing techniques may be implemented to help improve image quality.

Of course, these and/or other connectomics techniques may be used in connection with different embodiments of this invention.

Once the wiring diagrams are prepared, the neurons in the target regions may be enumerated, and the connections therebetween may be identified. Computer vision techniques may be used to help identify neurons in the wiring diagram, and/or their interconnections. A graph or other computer-implemented data structure may store such data. The graphs for the various members of the second sub-group may be compared to one another, and optionally, outliers may be excluded in step S210. Outliers may be identified, for example, by looking for the presence or absence of particularly high or low connection neurons, by implementing clustering techniques and then searching for the presence or absence of clusters, etc.

Activity in the wiring diagrams is recorded in step S212 as a sub-group is made to participate in the event to be studied, and/or activity in the brain is recorded and possibly matched with the wiring diagrams. The sub-group here may be a third sub-group, separate from the second sub-group, e.g., in the event that the second sub-group data is used as a baseline to which subsequently obtained data may be compared (e.g., in a search for changed connections, etc.). However, in certain example embodiments, the sub-group used here may be the second sub-group, e.g., if the wiring diagrams can be generated at the same time as the activities in the brain are being monitored and recorded. The activities monitored may include, for example, identifications as to transmitting and receiving neurons, neuron pairs or tuples that seem to be communicating with one another, the amount or intensity of the electrical signals generated therebetween, etc. As noted above, neural activity creates a perceivable electromagnetic field. Characteristics of this field may be determined, e.g., such that it is possible to quantify the size and shape of the overall field, and the intensity of the field at various different locations. The overall field may be decomposed or viewed as smaller fields to facilitate the pinpointing of active neurons contributing to the field, as well as the intensities at which those individual active neurons are operating. Intensity differences across the overall field or the smaller representations thereof may be located, as well. This type of decomposition may provide data in a format that is easier to subsequently process. As alluded to above, these electrical signals may be measured in connection with the wiring diagrams, or may be measured separately and later optionally mapped up with the wiring diagrams, e.g., based on positional relationships regarding where the electrical signals are initiated from and transmitted to, possibly taking into account both neural nodes and pathways.

In step S214, patterns in the presence and/or intensity of the signals in the wiring diagrams may be identified. This pattern matching may take place for both an individual test subject, as well as across multiple test subjects. For example, pattern identification or clustering techniques may be leveraged to determine which nodes, node connections, electrical signals, etc., seem to be associated with the event to be studied for a given individual. Those individual patterns may be cross-referenced among like data gathered from the other test subjects to improve the likelihood that there is a connection between the observed field and the event being studied. Ultimately, over time, key nodes and/or areas may be associated with a particular event being studied.

In step S216, the patterns may be associated with attributes of the members of the sub-group from which the measurements were taken. These attributes may include any demographic or personal information concerning the subject including, for example, age, gender, race, ethnicity, country of origin, place of upbringing, rural/suburban/urban living conditions, education levels, IQ, known genetic or acquired neural or other diseases or disorders, etc. The patterns and attributes may be stored in a database in step S218.

The end result may be a database or databases of how various events are processed. As one example, a database may be generated so as to include information regarding how children learn to do mathematics, with separate entries being provided for different levels of mathematics (e.g., for basic arithmetic, algebra, calculus, differential equations, etc.), and for different demographic and/or other groups of people such as, for example, for males and females, different age children, children from different geographic regions of this or some other country, etc. As another example, a database may be generated so as to include information regarding how seizures take place, etc. As still another example, a database of specific “lessons” may be developed. Such specific lessons may include, for example, phonics, long division, state capitals, muscle control/movement, etc.

It will be appreciated that pre- and post-treatment wiring diagrams may be stored in the database for the subjects. This may help with identifying whether a potential new subject qualifies for treatment, and/or for assessing whether that treatment likely will be successful. The database also may be updated to include effectiveness information, e.g., if the subjects come back over time, and if data is kept regarding any lasting positive or negative effects. For example, qualitative or quantitative data can be obtained, e.g., based on self-reporting or family reporting; observational studies; performance on IQ, memory, skills, or other tests; etc. This may make it possible to determine whether a particular skill was mastered or skill level was at least improved, to assess the extent of Alzheimer's degradation and/or determine whether it was slowed or stopped, etc.

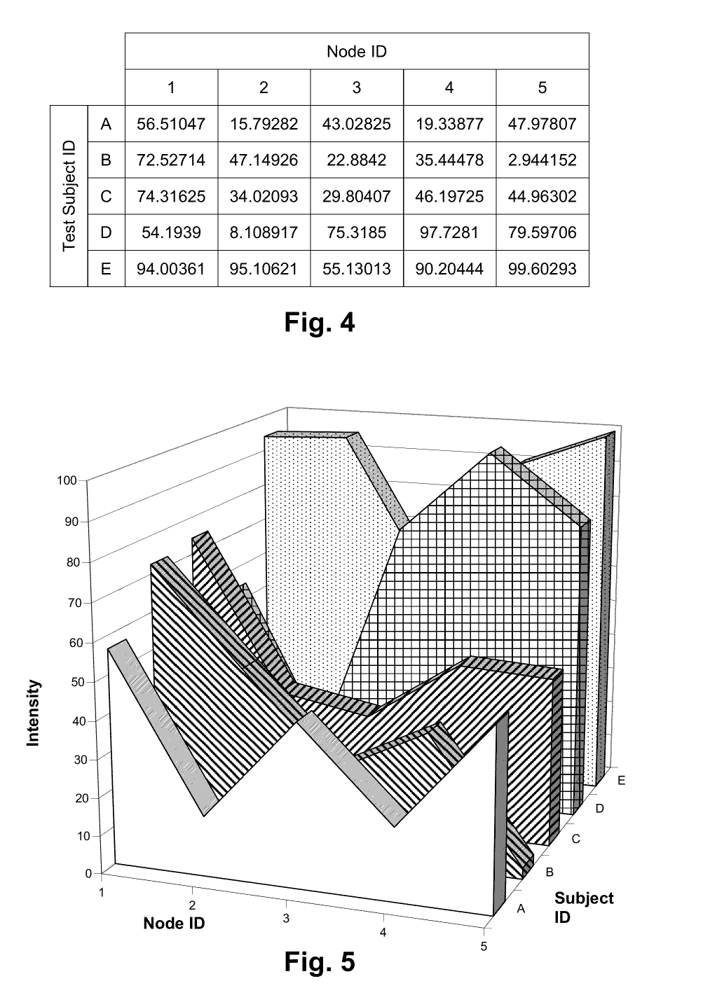

As indicated above, a wide variety of electrical and/or other data can be gathered for test subjects, e.g., in creating the various databases. FIG. 4 is a table showing intensity measurements of various nodes for five test subjects at a given point in time, showing one type of data and how it can be recorded in connection with certain example embodiments, and FIG. 5 a graph plotting the FIG. 4 data. The FIG. 4 table basically lays out the electrical intensities generated at nodes 1-5 for test subjects A-E. Many tables such as those shown in FIG. 4 may be gathered over a predetermined time period, e.g., as a person performs a task of interest, is exposed to a stimulus, experiences a seizure, etc. The data in such tables may be auto-correlated and/or cross-correlated. This may help illustrate whether there are patterns in how the nodes interact on an individual basis over time, how they interact with one another, and/or whether such correlations are common across different test subjects.

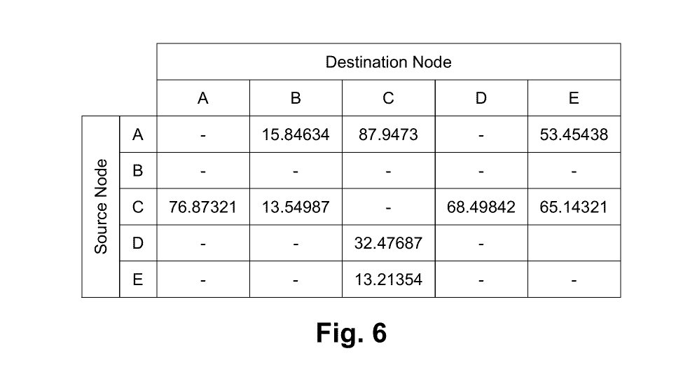

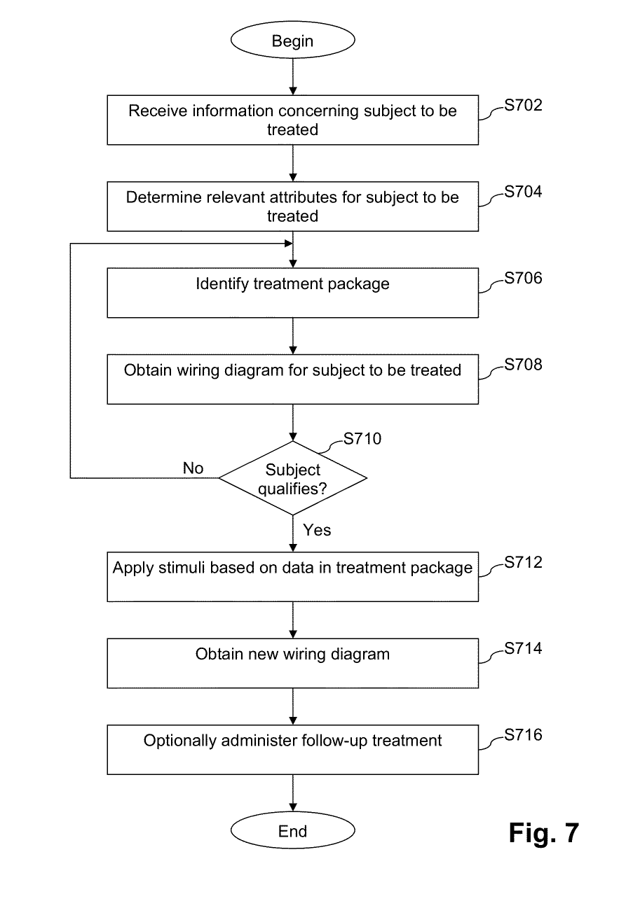

FIG. 6 is an example data structure showing connections between nodes at a given time and for a given test subject, thereby illustrating another type of data that may be gathered and analyzed in connection with certain example embodiments. The FIG. 6 table may represent a directed graph, with connections between nodes being indicated by a non-zero intensity value. Again, many tables such as those shown in FIG. 4 may be gathered over a predetermined time period, e.g., as a person performs a task of interest, is exposed to a stimulus, experiences a seizure, etc. The graph may be traversed using known algorithms, e.g., to determine whether there are any clusters, cliques of neurons that seem to be activated, etc. Patterns in electrical intensity values also may be analyzed using flow analysis techniques. This may help reveal how an electrical field is setup and progresses over time for a given test subject. As above, data from multiple test subject may be compared.

In certain example embodiments, a computer system may be trained to recognize patterns in neural activity based on repeated testing as subjects are asked to perform the same task, over and over. For instance, subjects may be asked to perform various simple addition problems over and over again. The computer system may monitor the neurons and pathways that are activated during these activities and, once a threshold amount of data has been collected, determine that a particular subset of neurons and pathways are associated with the task, at least in “normal” or “properly functioning” brains. Then, as explained in greater detail below, these neurons can be stimulated in test subjects via electrical excitation to cause them to more easily handle similar such activities, and/or even gain the benefits of such practice over time.

Once the neurons and their behaviors are mapped and associated with a given activity, it becomes possible to simulate or stimulate their behaviors. To facilitate learning, for example, it may be possible to set up electromagnetic fields that target nodes associated with improved memory, thereby artificially triggering neural responses. In certain embodiments, electromagnetic fields associated with memory can be triggered as an individual receives more traditional pedagogical instruction, thereby leading the subject to “get it the first time around” or to help compensate for learning disabilities or the like. By improving the ability to remember, it is possible to increase the capacity for learning, even where subjects may have innate disadvantages in this respect (e.g., because of autism, ADD or ADHD, etc.). In still other examples, particular lessons may be transmitted electrically.

With respect to subjects found to be suffering from certain mental diseases or disorders, or other diseases or disorders that can be linked to a nervous system abnormality, it may be possible to artificially simulate, stimulate, or even repair “missing links,” cause non-functioning synapses to process information correctly, train nerves to respond, simulate developmental movements, etc. This may have advantages in preventing epilepsy and MS, or quickly treating epileptic events, MS flare-ups, etc. Neural degradation caused by botulism, Parkinson's, Alzheimer's, etc., may be slowed, halted, or even reversed, in some cases, by identifying misfiring synapses, non-functional neurons, etc., and correspondingly treating them, e.g., by repairing connections, artificially creating or stimulating signal transmission, encouraging memory, etc.

Because some forms of depression involve chemical imbalances in the brain, electromagnetic fields may be setup to encourage the release of other chemical as a counter-balance or to discourage the emission of the overly active chemical(s) in the first place. This form of treatment may be advantageous because it may bypass chemical or pharmaceutical treatment routes and instead encourage direct electrical stimulation of the relevant neurons, or at least more direct electrochemical stimulation of the relevant neurons (e.g., in the event that the electrical signal causes a chemical to be released, etc.).

If enough data is gathered at a fine enough detail, it even becomes possible to simulate intake of “hard facts” or transmit “feelings.” Cochlear implants already provide direct electrical stimulation to the acoustic nerve, and other senses can be stimulated in similar ways, particularly if electrical stimulation is provided to nerves in a way to which the brain is naturally predisposed to understand. It also becomes possible to compensate for genetic, environmental, or developmental issues, e.g., by turning on or off neural pathways.

FIG. 7 is a flowchart showing a treatment process in accordance with certain example embodiments. In step S702, information concerning the subject to be treated is received. This information may include, for example, information concerning the objectives of the treatment (e.g., memory or concentration improvement, specific subject or subject matter “tutoring,” treatment for a mental disorder or disease, nerve repair, etc.), medical history of the subject (e.g., whether the subject suffers from any known mental or other diseases or disorders, has undertaken any previous treatments, suffered nerve damages, etc.), demographic data pertaining to the patient (e.g., age, race, ethnicity, educational level, IQ, etc.), and/or the like. In the case of human subjects, for example, such information may be received via a paper or electronic patient intake or subject inquiry form. It will be appreciated that not all received patient information will be relevant to all treatments. For instance, IQ may not be necessary information when determining whether a subject qualifies for Parkinson's treatment or nerve reconditioning (e.g., after an accident leaves the subject partially paralyzed), but may be valuable data when determining whether a subject is having memory or concentration issues. Thus, in step S704, the relevant attributes for the subject to be treated are determined from the larger set of gathered data received in step S702.

Once this data is ascertained, a candidate treatment package is identified in step S706, e.g., by comparing the data concerning the subject to data about the test groups. If there is a seeming match, then a wiring diagram for the subject may be obtained in step S708. This baseline wiring diagram may be examined and optionally compared to wiring pre-treatment wiring diagrams of the patients, as there may be some otherwise “hidden” incompatibilities revealed only by obtaining and possibly subsequently comparing wiring diagrams. For instance, wiring diagrams of the subject to be treated may reveal that there are no similarities to the test group subjects, that there is other or perhaps unappreciated damage to presumed key neurons or neuron linkages, etc. Thus, the determination as to whether the subject qualifies for treatment in some cases may be based at least in part on a comparison between a pre-treatment connectome in an area proximate to a key treatment zone, and a pre-treatment connectome associated with the template map stored in the database, with the key treatment zone being specified in the database.

If the subject does not qualify for treatment, then the process may return to step S706, e.g. to search for an alternate treatment regimen. However, if the patient does qualify for the treatment, then stimuli may be applied in step S712 based on the selected treatment package. The treatment may be applied via contact or non-contact means, and the voltage(s) applied may be sufficient to set up electromagnetic or other fields such as those observed during the test subjects' treatments in certain example embodiments. This may, in turn, result in the transmissions of data from neuron-to-neuron, as expected, either directly (e.g., via direct electrical triggering of synaptic actions) or by triggering responses in neurons that, in turn, release chemicals involved in electrochemical communications. These approaches may provide a much faster acting and potentially more targeted response, while also potentially reducing side-effects, as compared to chemical or pharmaceutical treatments associated with certain drugs (e.g., various classes of anti-depressants, anti-psychotic agents, ADD-offsetting drugs like Ritalin and Adderall, etc.).

With respect to certain contact means embodiments, it is noted that electrodes may be implanted into the subject's brain, e.g., at or proximate key neurons, areas of the brain where the neurons are located or that are believed to be primarily associated with the desired treatment outcome, gateways to such areas, etc. The implantation of electrodes may enable remote or selective triggering. For instance, the onset of a seizure may be automatically detected by a sensor based on inputs received from the implanted electrode and/or associated circuitry, and/or the subject may feel the onset of a seizure, stroke, or the like. In such cases, small charges from the electrodes can be triggered, e.g., to help mitigate such events. Similarly, a subject may self-administer treatments or have treatments administered during school or other learning functions, during social settings, etc., to help facilitate learning and/or other activities.

Non-contact means may also be used in certain example embodiments. For instance, electromagnetic fields can be projected onto corresponding locations or gateways, e.g., using electrode arrays disposed around the subject's head, by creating coherent holographic projections in the proper frequency range, etc. For certain example non-contact means, the subject may be immobilized or even sedated during the treatment so that the electromagnetic fields can be created at a sufficiently high resolution with a sufficiently low likelihood of “missing” the intended targets. In other example non-contact embodiments, a hat or other device with electrode arrays may be worn by the patient. In certain cases, treatments may take place in a clinical environment. However, in other cases, treatments may take place at home (e.g., while the subject sleeps, needs to interact with others, etc.), at a school (e.g., while the subject is engaged in a learning activity, needs to concentrate, etc.), and/or at other places and/or times.

In certain example embodiments, the charges delivered may be above, at, or below the threshold that elicits a neuronal discharge that occurs after termination of a stimulus. Thus, certain example embodiments may completely replace neuronal discharges, or simply provide a modest “boost” to perhaps help the neurons to function normally or to help train the neurons to function normally. The latter treatment technique may be practiced either to promote natural responses that otherwise might occur more slowly or not at all, and/or to rehabilitate damaged neurons and/or pathways. A therapeutic or rehab-type level may be between about 1.0-2.0 mA in certain cases, although higher or lower charges may be warranted in different situations, e.g., in dependence on the electrical field measurements discussed above, the area of the brain involved, the extent of damage suffered, and/or the like. Square, sine, or other type waveforms may be used, although generally rectangular waveforms are believed to be compatible with certain neural activities. Observed amperages may be lowered by a predetermined percentage in certain example embodiments.

Once the treatment is completed, in step S714 a new wiring diagram optionally may be obtained. This new wiring diagram may be compared to wiring diagrams in the database to suggest whether the treatment was successful, whether abnormalities occurred, etc. For example, in certain example embodiments, a post-treatment connectome may be obtained for the subject in the key treatment area. The post-treatment connectome for the subject may be compared with a post-treatment connectome associated with the template map stored in the database. The effectiveness of the treatment may be gauged based on the comparison.

As above, effectiveness information may be assessed and stored for the patient. The pre- and post-treatment wiring diagrams may be added to the database if they are generally in conformance with that which is expected, optionally together with relevant attributes of the subject. This may help build the database and/or improve its accuracy over time.

Follow-up treatments optionally may be administered, when and as needed, e.g., in step S716. Subsequent wiring diagrams may be gathered, and effectiveness information optionally may be assessed and stored, e.g., after each follow-up treatment. This information also may be added to the database, e.g., as discussed above.

Although certain example embodiments have been described as targeting individual neurons, neuron clusters, etc., for electrical stimulation, the example embodiments described herein may have broader applications. For instance, once a wiring diagram is developed and areas of brain activity associated with particular events are identified, the pathways leading to these areas may be stimulated instead, e.g., to remove “upstream blockages” that might occur. For instance, spatial memory can be enhanced by electrical stimulation of the entorhinal region, which is a specific site within the medial temporal lobe and a gateway into the hippocampus. Other types of memory may be enhanced by targeting respective gateways into the hippocampus and/or other areas of the brain associated with such activities.

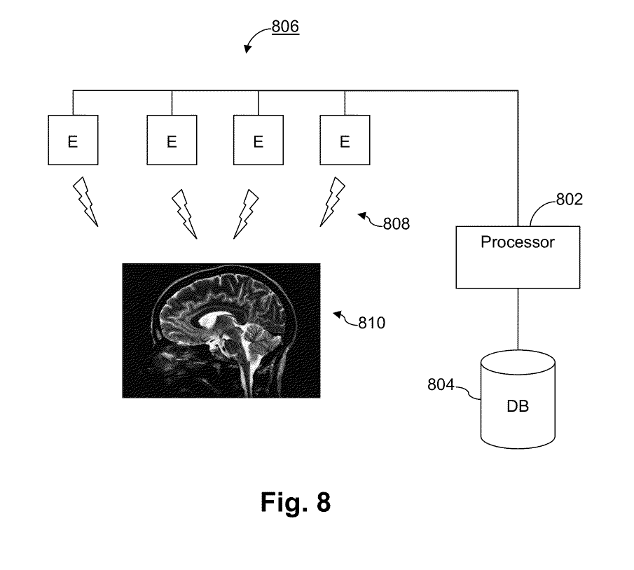

In certain example embodiments, a treatment device is provided, e.g., as shown in FIG. 8. A processor 802 is configured to read a template map from a database 804 of template maps, with each template map indicating neurons and/or neural pathways to be electrically excited, and an intensity of excitation for each, at specific times over a predefined treatment time period. The template map represents a desired treatment objective for a subject and the treatment map being customized for associated subject-specific characteristics. An electrode array 806 includes one or more electrode E and is configured to electrically stimulating (e.g., by setting up an electromagnetic field via signals 808) the subject's brain 810 in accordance with the selected template map, based on instructions from the processor.

A detector may be provided and configured to detect onset of a neural abnormality. The neural abnormality may correspond to, for example, to a chemical imbalance, epileptic seizure, or triggering of multiple sclerosis. The detector may be configured to communicate with the processor of the device in order to selectively activate the electrode array in response to the detected onset of the neural abnormality.

As alluded to above, the electrode(s) may be implanted in a brain or external to the subject's body. In the latter regard, an article may support an electrode array, and this article may be worn by the subject.

Although certain example embodiments have been described in connection with human subjects, the example embodiments described herein can be used in connection with any vertebrate or invertebrate include a brain or large cluster of neurons. Indeed, in certain example embodiments, it may be advantageous to perform tests of mice, monkeys, or other animals, e.g., in order to determine likely parts of the brain that are associated with certain activities such as, for example, learning, memorizing, etc., as well as certain diseases or disorders such as autism, epilepsy, Alzheimer's, Parkinson's, etc.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A brain activity monitoring method, the method comprising:
selecting a group of test subjects such that the test subjects have at least some personal attributes in common;
asking or allowing the group of test subjects to perform a common event over a predetermined time period;
measuring electrical signals generated by individual test subjects as each performs the common event;
attributing the measured electrical signals to neurons and/or neural pathways of the test subjects' brains to develop a time-indexed map of neural activity for the common event for each said test subject, the attributing including assigning location and intensity values to each measured electrical signal at regular intervals over the predetermined time period during which the common event was performed;
comparing the maps to one another and discarding outlier maps, locations, and intensity values; and
storing to a database of a computer system one or more maps as a template time-indexed map of neural activity for the common event.

2. The method of claim 1, further comprising:
identifying key neurons or clusters of neurons associated with the common event; and
identifying one or more gateways in the brain to the identified key neurons or clusters of neurons.

3. The method of claim 1, further comprising:
monitoring brain activity using fMRI and/or EEG hardware for a first subset of test subjects as the first sub-set of test subjects performs the common event; and
identifying one or more portions of the brain that appear to be associated with the common event based on output from the fMRI and/or EEG hardware,
wherein the measuring of the electrical signals is focused on the one or more identified portions of the brain, the measuring being performed on a second subset of test subjects following said monitoring.

4. The method of claim 3, wherein the first and second subsets of test subjects are separate subsets.

5. The method of claim 4, wherein at least some test subjects are twins, a first twin being placed in the first subset and a second twin being placed in the second subset.

6. The method of claim 3, further comprising building connectomes for the one or more portions of the brain.

7. The method of claim 1, wherein the personal attributes include one or more of: age, education level, ethnicity, gender, mental disease and/or disorder, nervous system malady, and race.

8. The method of claim 1, wherein the common event is selected from the group consisting of: reading, writing, performing mathematical operations, memorizing, suffering a seizure, and engaging a muscle or group of muscles.

9. The method of claim 1, wherein the database further includes personal attributes associated with the template time-indexed map of neural activity for the common event.

10. A brain activity monitoring system, comprising:
measuring means for measuring electrical signals generated by individual test subjects in a group of test subjects as each performs a common event over a predetermined time period, the test subjects being selected so that they have at least some personal attributes in common;
a non-transitory storage location for storing data generated by the measuring means;
a computer system including at least one processor and a memory and being configured to:
read the data generated by the measuring means stored in the storage location,
attribute the measured electrical signals to neurons and/or neural pathways of the test subjects' brains to develop a time-indexed map of neural activity for the common event for each said test subject, the attributing including assigning location and intensity values to each measured electrical signal at regular intervals over the predetermined time period during which the common event was performed, and
compare the maps to one another and discard outlier maps, locations, and intensity values; and
a database in communication with the computer system, the database being configured to store one or more maps as a template time-indexed map of neural activity for the common event.

11. A method of stimulating a subject's brain, the method comprising:
selecting a template map of neural activity from a database comprising a plurality of template maps,
the template map being selected based at least in part on a desired treatment objective and subject-specific characteristics of the subject,
the template map indicating neurons and/or neural pathways to be electrically excited, and an intensity of excitation for each, at specific times over a predefined treatment time period; and
electrically stimulating the subject's brain in accordance with the selected template map.

12. The method of claim 11, further comprising determining whether the subject qualifies for treatment prior to the electrical stimulating.

13. The method of claim 12, wherein the determination as to whether the subject qualifies for treatment is based at least in part on a comparison between a pre-treatment connectome in an area proximate to a key treatment zone, and a pre-treatment connectome associated with the template map stored in the database, the key treatment zone being specified in the database.

14. The method of claim 13, further comprising:
obtaining a post-treatment connectome for the subject in the key treatment area;
comparing the post-treatment connectome for the subject with a post-treatment connectome associated with the template map stored in the database; and
gauging effectiveness of the treatment based on the comparison.

15. The method of claim 11, wherein the subject-specific characteristics of the subject include one or more of: age, education level, ethnicity, gender, mental disease and/or disorder, nervous system malady, and race.

16. The method of claim 11, wherein the desired treatment objective includes one or more of: an educational goal, memory/recall improvement, nerve reconditioning, and mitigation and/or correction of neural degradation.

17. The method of claim 11, further comprising during said electrical stimulating, setting up an electromagnetic field in/on the subject's brain in accordance with the template map via electrodes external to the subject's body.

18. The method of claim 11, wherein the intensities in the template map are reduced from measured values by a predetermined percentage.

19. The method of claim 17, wherein the intensities in the template map are reduced to levels below threshold levels for directly initiating synaptic responses.

20. A treatment device, comprising:
a processor configured to read a template map indicating neurons and/or neural pathways to be electrically excited, and an intensity of excitation for each, at specific times over a predefined treatment time period, the template map representing a desired treatment objective for a subject and the treatment map being customized for associated subject-specific characteristics;
an electrode array including one or more electrodes, configured to electrically stimulating the subject's brain in accordance with the selected template map, based on instructions from the processor.

21. The treatment device of claim 20, wherein the electrode array is implanted in the subject's brain.

22. The treatment device of claim 20, further comprising a detector configured to detect onset of a neural abnormality, the detector being further configured to communicate with the processor in order to selectively activate the electrode array in response to the detected onset of the neural abnormality.

23. The treatment device of claim 22, wherein the neural abnormality corresponds to a chemical imbalance, epileptic seizure, or triggering of multiple sclerosis.

24. The treatment device of claim 20, wherein the electrode array is external to the subject's body.

25. The treatment device of claim 24, further comprising an article supporting the electrode array and adapted to be worn by the subject.
